(19) United States
(12) Patent Application Publication
(10) Pub. No.: US 2009/0239281 A1
(43) Pub. Date: Sep. 24, 2009

(54) Moving A Small Object in A Direction

(75) Inventors: Osman Kibar, San Diego, CA (US); Mirianas Chachisvilis, San Diego, CA (US); Eugene Tu, San Diego, CA (US); Andrew Senyei, La Jolla, CA (US)

Correspondence Address: FISH & RICHARDSON PC, P.O. BOX 1022, MINNEAPOLIS, MN, 55440-1022, US

(73) Assignee: Dynamic Connections, LLC, San Diego, CA (US)

(21) Appl. No.: 12/407,380

(22) Filed: Mar. 19, 2009

Related U.S. Application Data
(60) Provisional application No. 61/038,573, filed on Mar. 21, 2008.

(51) Int. Cl. C12N 13/00; C12N 1/00; C12N 1/20; C12N 7/01; G01N 33/48; G01N 33/92; C12N 9/00

(52) U.S. Cl. 435/173.1; 435/243; 435/252.1; 435/235.1; 436/86; 436/94; 436/71; 435/183

(57) ABSTRACT

For directional motion of a small object, a combination of at least two different properties is imparted to the object. At least one of the properties being responsive to an external influence to produce a force or torque acting on the object. At least another of the properties is responsive to the force or torque to cause a directional motion of the object.

This application has the benefit of the filing date of U.S. provisional application 61/038,573, filed Mar. 21, 2008, and incorporated in its entirety here by reference.

U.S. patent application Ser. No. 12/103,281, filed Apr. 15, 2008, is incorporated in its entirety here by reference.

U.S. provisional application 60/987,674, filed Nov. 13, 2007, is incorporated in its entirety here by reference.

### BACKGROUND

This description relates to moving a small object in a direction.

Small objects can be moved in specific directions by applying an external force or torque.

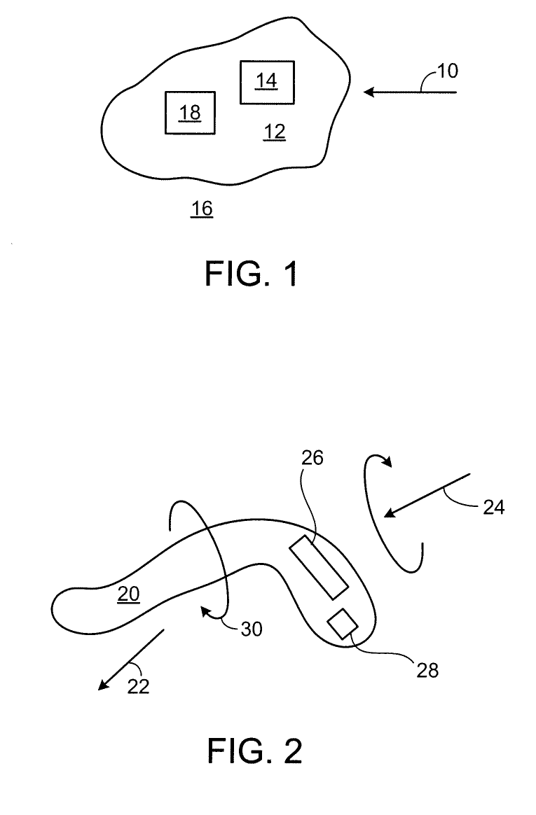

In some cases, the external force acts on a property of the object that is inherent to the object, renders the object susceptible to the force, and differs from a property of the medium in which the object is to be moved. For example, as shown in FIG. 1, in dielectrophoresis, an oscillating electric field (the external force 100) is applied to a cell (the object 12). The cell has a value of dielectric constant (a property 14) that is natural (inherent to the cell), makes the cell susceptible to the force, and is different from the value of dielectric constant of the fluid (the medium 16) around it.

In some cases, a property 18 that makes an object susceptible to an external force is not inherent to the object, but instead is imparted to it. For example, a charge (the property) can be imparted to a denatured protein by attaching negatively charged SDS molecules to it. Electrophoresis can then be performed by applying an electric field.

In some cases, for example a chiral molecule 20 as shown in FIG. 2, the object can be moved in a direction 22 by applying a rotating electric field 24 (the external influence). The rotating field acts on an electric dipole moment 26 of the molecule (the dipole moment being a first inherent property of the chiral molecule), causing the molecule to rotate. The chirality 28 of the molecule (its chirality being a second, inherent property) translates the molecule's rotation 30 into motion in a direction 22. The direction 22 depends on the molecule's handedness (i.e., its chirality).

### SUMMARY

In general, in an aspect, for directional motion of a small object, a combination of at least two different properties is imparted to the object, at least one of the properties being responsiveness to an external influence to produce a force or torque acting on the object, at least another of the properties being responsiveness to the force or torque to cause a directional motion of the object.

Implementations may include one or more of the following features. Prior to the combination of the at least two properties being imparted to the object, no directional motion of the object is produced in response to the external influence. Prior to the combination of the at least two properties being imparted to the object, any directional motion of the object in response to the external influence is less than after the imparting. Neither of the properties is inherent in the object.

The object includes a cell, for example, a cell that is abnormal, dead, diseased, or drugged or exposed to other chemicals. The object includes a virus. The object includes, for example, a bacterium, a molecule, a biomolecule, a protein (which has, for example, a post-translational modification), a peptide, an enzyme, a DNA or RNA molecule, which may include an epimer, a polymer, a bead, a nanoparticle, an organelle, a liposome, a vesicle, a biomolecular aggregate, a molecular aggregate, or a spore.

The imparting includes attaching at least one other object, such as an antibody. The imparting includes adding another object. The other object includes a transfected gene. The imparting includes removing a part of the object. The imparting includes causing a reaction on the object. The reaction, for example, is molecular, cellular, or chemical.

The external influence includes a force field, such as electric, magnetic, or gravitational. The gravitational field is centrifugal. The field includes a pressure field or an acoustic field. The force field is static, oscillating, or rotating. The external influence includes a reaction. The reaction is chemical, such as rotatory, molecular, or cellular. The external influence includes a kinetic force, such as electroosmotic, electrophoretic, or one based on dielectrophoretic, optophoretic, or thermophoretic. The external influence includes a gradient, such as a chemical gradient or a field gradient. The other property includes chirality.

One of the properties may be imparted in the form of a dipole, such as an electric dipole or a magnetic dipole. The external influence may be a rotating electric field or a rotating magnetic field.

These and other aspects and features, and combinations of them, may be expressed as methods, apparatus, systems, means for performing functions, program products, and in other ways.

Other advantages and features will become apparent from the following description and from the claims.

### DESCRIPTION

FIGS. 1, 2, 3, and 4 are schematic views of objects and external influences.

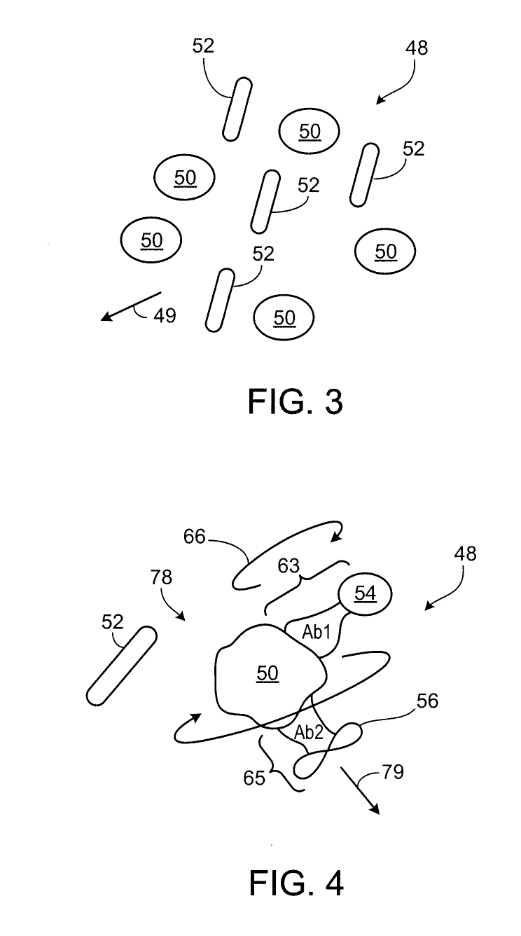

As shown in FIG. 3, a small object 40 can be moved in a direction 42 by (for example) an external force or torque 44 that operates on the object by means of at least two properties of the object (at least two of which 46, 48 are not inherent but have been imparted to the object). The motion in direction 42 occurs only if the object has both of the imparted properties, like an AND-logic gate, the output of which is 1 (corresponding to directional motion) only if the two inputs (the imparted properties), both have a value 1 (i.e., are present). There can be more than two imparted properties, and the object can also have one or more inherent properties.

For example, as shown in FIG. 3, assume that a biological sample 48 includes two different types of cells, one population of target cells 50 (TC's) and a second population of non-target cells 52 (NTC's), and the goal is to obtain an enriched population of TC's by moving 49 TC's from the sample and leaving NTC's behind.

As shown in FIG. 4, assume that we have supplies of (a) two antibodies (Ab1 and Ab2) that both have a stronger affinity to TC's than to NTC's, e.g., they preferentially bind to TC's and much less so to NTC's, (b) magnetic beads 54 that are super-paramagnetic, i.e., they exhibit magnetism (or they acquire a magnetic dipole moment) in the presence of a magnetic field, and (c) small objects 56 (e.g., ˜1-10 μm diameter, or 0.1-1 μm diameter), either manufactured or natural that have a certain chirality (i.e., right-handed or left-handed, so let's assume all left-handed in this case). We sometimes call the small objects 56 propellers.

As shown in FIG. 4, we attach each Ab1 to a magnetic bead to form one complex 63, and each Ab2 to an artificial propeller 56 to form a complex 65, and we put both complexes (either sequentially or simultaneously) into the sample 48 that contains both TC's and NTC's. (In FIG. 4, we have only shown a single TC and a single NTC. Each Ab1-magnetic bead complex 63 will attach to a TC in a specific manner with a relatively higher probability or to an NTC in a non-specific manner with a relatively lower probability. For simplicity of discussion, assume that the Ab1-magnetic bead complexes will only bind to TC's and that the non-specific binding to NTC's is negligible.

Similarly, assume for simplicity that the Ab2-propeller complex 65 binds only to TC's (and not to NTC's).

When an Ab1-magnetic bead complex binds to a TC, the magnetic bead will impart a magnetic dipole to the TC; an Ab2-propeller complex, in turn, will impart chirality to the TC.

We then apply a rotating magnetic field 66 to the sample, causing all magnetic beads, whether bound to TC's or not, to rotate with the magnetic field.

Any magnetic bead that is not bound to a cell or any complex of a bead bound to a cell that does not also have a propeller bound to it will not have any chirality, and therefore, will not move in a particular direction, in other words, it will not experience any directional motion.

Conversely, a propeller that is not bound to a cell or any complex of a propeller that is bound to a cell that does not also have a magnetic bead bound to it, will not have any magnetism, and the rotating magnetic field will have no effect on it. Even though the complex has chirality, it lacks magnetism to convert the external influence (the rotating magnetic field, in this case) into a force or a torque to cause it to rotate (and in turn be moved in a direction).

Only a cell 78 that has both a magnetic bead and a propeller attached to it (using Ab1 and Ab2, respectively, in this example) will rotate in response to a rotating magnetic field, and move in a direction 79 as the rotation is converted into directional motion (as a result of the chirality imparted to the cell by the propeller). Additional information about the use of rotation and chirality for propeller motion can be found in U.S. patent application Ser. No. 12/103,281, filed Apr. 15, 2008, and incorporated in its entirety here by reference.

By imparting two properties, e.g., magnetism and chirality, that are not inherent to a small object (e.g., a cell), e.g. by attaching two other objects (one with magnetism, the other with chirality), an external influence (e.g., a rotating magnetic field) can be converted into a torque on the cell to rotate it and the rotation be used to move the cell in a particular direction (that is determined by the handedness of the propeller). In this example, both magnetism and chirality are required to generate the directional motion; if one is missing, there's none (without magnetism, the cell does not rotate, and without chirality, it is not propelled).

Using this combination of properties and an external influence, for example, target cells can propel themselves to a specific location in the container (using the specific binding of antibodies appropriate for those cells), leaving the non-target cells behind. Because both antibodies are selected to be ones that bind to the target cell and not to the non-target cell, the enrichment factor can be greatly enhanced. Furthermore, losses due to harsh and the use of possibly multiple extraction steps can be avoided, and the yield of such an enrichment process can be high. Yield loss due to de-bulking, for example, can be reduced.

Other examples are also within the scope of the claims.

The object need not be a cell. It could be a virus, bacteria, molecule, biomolecule, protein, peptide, enzyme, DNA, RNA, small molecule, polymer, bead, nanoparticle, organelles, liposome, vesicle, biomolecular aggregate, molecular aggregate, or spore, or any other small particle or object.

The cells may be modified to be abnormal, dead, diseased, and/or drugged; the proteins may have certain PTMs (post-translational modifications, e.g., glycosylated, phosphorylated); DNA and/or RNA molecules may be epimers.

The properties can be imparted to the objects in a wide variety of different ways. For example, an object (e.g., an antibody or a transfected gene) can be attached to or added to the target object. A portion of the target object can be removed. A reaction can be caused on the object (e.g., chemical, molecular or cellular) to change one of its properties.

The external influence may be: a field (electric, magnetic, electromagnetic, gravitational/centrifugal, pressure/acoustic/ultrasound), a reaction (e.g. chemical/rotatory chemistry, molecular, cellular), a kinetic force (e.g. electroosmotic, electrophoretic, dielectrophoretic, optophoretic, thermophoretic), or a gradient (e.g. chemical, field).

The property of the object to convert the external influence into a force or torque on the object may be a property that interacts with the external influence. For example, the property may be a permanent or induced electric dipole moment that interacts with an electric field; a permanent or induced magnetic dipole moment that interacts with a magnetic field; permittivity or permeability with respect to an electromagnetic field; mass (or buoyancy) with respect to a gravitational field; and so on for other external influences, including the ones mentioned above.

The second property to convert the force or torque on the object into directional motion of the object may be chirality, as explained in the examples, or other properties that produce directional motion.

We have used the word inherent to refer, for example, to a property that exists in an object in its naturally occurring form, or in the object in a form in which it is initially manufactured or altered or contemplated.

In some cases, the two properties can be imparted to the target object by a single action or a single other object. For example, the propelling property and the force receiving property can be embodied in a single object which is then attached to the target object.

In some cases, the object may have the two properties and the effect of the two properties for purposes of causing directional is enhanced by imparting the two properties again or in greater amount or in a second version, for example, to the object.

1. A method for use in directional motion of a small object, the method comprising,
imparting to the object a combination of at least two different properties,
at least one of the properties being responsive to an external influence to produce a force or torque acting on the object,
at least another of the properties being responsive to the force or torque to enhance the directional motion of the object.

2. The method of claim 1 in which, prior to the combination of the at least two properties being imparted to the object, there is no directional motion of the object in response to the external influence.

3. The method of claim 1 in which, prior to the combination of the at least two properties being imparted to the object, the directional motion of the object in response to the external influence is less than after the imparting.

4. The method of claim 1 in which neither of the properties is inherent in the object.

5. The method of claim 1 in which the object comprises a cell.

6. The method of claim 5 in which the cell is abnormal.

7. The method of claim 5 in which the cell is dead.

8. The method of claim 5 in which the cell is diseased.

9. The method of claim 5 in which the cell is drugged or exposed to other chemicals.

10. The method of claim 1 in which the object comprises a virus.

11. The method of claim 1 in which the object comprises a bacterium.

12. The method of claim 1 in which the object comprises a molecule.

13. The method of claim 1 in which the object comprises a biomolecule.

14. The method of claim 1 in which the object comprises a protein.

15. The method of claim 14 in which the protein has a post-translational modification.

16. The method of claim 1 in which the object comprises a peptide.

17. The method of claim 1 in which the object comprises an enzyme.

18. The method of claim 1 in which the object comprises a DNA molecule.

19. The method of claim 18 in which the DNA molecule comprises an epimer.

20. The method of claim 1 in which the object comprises an RNA molecule.

21. The method of claim 20 in which the RNA molecule comprises an epimer.

22. The method of claim 1 in which the object comprises a polymer.

23. The method of claim 1 in which the object comprises a bead.

24. The method of claim 1 in which the object comprises a nanoparticle.

25. The method of claim 1 in which the object comprises an organelle.

26. The method of claim 1 in which the object comprises a liposome.

27. The method of claim 1 in which the object is a vesicle.

28. The method of claim 1 in which the object comprises a biomolecular aggregate.

29. The method of claim 1 in which the object comprises a molecular aggregate.

30. The method of claim 1 in which the object comprises a spore.

31. The method of claim 1 in which the imparting comprises attaching at least one other object.

32. The method of claim 31 in which the other object comprises an antibody.

33. The method of claim 1 in which the imparting comprises adding another object.

34. The method of claim 33 in which the other object comprises a transfected gene.

35. The method of claim 1 in which the imparting comprises removing a part of the object.

36. The method of claim 1 in which the imparting comprises causing a reaction on the object.

37. The method of claim 36 in which the reaction is molecular.

38. The method of claim 36 in which the reaction is cellular.

39. The method of claim 36 in which the reaction is chemical.

40. The method of claim 1 in which the external influence comprises a force field.

41. The method of claim 40 in which the force field is static.

42. The method of claim 40 in which the force field is oscillating.

43. The method of claim 40 in which the force field is rotating.

44. The method of claim 40 in which the field comprises an electric field.

45. The method of claim 40 in which the field comprises a magnetic field.

46. The method of claim 40 in which the field comprises a gravitational field.

47. The method of claim 46 in which the gravitational field is centrifugal.

48. The method of claim 40 in which the field comprises a pressure field.

49. The method of claim 40 in which the field comprises an acoustic field.

50. The method of claim 1 in which the external influence comprises a reaction.

51. The method of claim 50 in which the reaction is chemical.

52. The method of claim 51 in which the chemical reaction is rotatory.

53. The method of claim 50 in which the reaction is molecular.

54. The method of claim 50 in which the reaction is cellular.

55. The method of claim 1 in which the external influence comprises a kinetic force.

56. The method of claim 55 in which the kinetic force is electroosmotic.

57. The method of claim 55 in which the kinetic force is based on electrophoretic.

58. The method of claim 55 in which the kinetic force is based on dielectrophoretic.

59. The method of claim 55 in which the kinetic force is based on optophoretic.

60. The method of claim 55 in which the kinetic force is based on thermophoretic.

61. The method of claim 1 in which the external influence comprises a gradient.

62. The method of claim 61 in which the gradient comprises a chemical gradient.

63. The method of claim 61 in which the gradient comprises a field gradient.

64. The method of claim 1 in which the other property comprises chirality.

65. The method of claim 1 in which one of the properties is imparted in the form of a dipole.

66. The method of claim 65 in which the dipole comprises an electric dipole.

67. The method of claim 65 in which the dipole comprises a magnetic dipole.

68. The method of claim 66 in which the external influence comprises a rotating electric field.

69. The method of claim 67 in which the external influence comprises a rotating magnetic field.

70. A method comprising
enhancing directional motion of a small object by applying an external influence to produce a force or torque acting on the object by operation of a first property that has been imparted to the object, the force or torque enhancing the directional motion of the object by operation of a second property that has been imparted to the object and includes responsiveness to the force or torque to cause the directional motion.

71. The method of claim 70 in which the small object is part of a first quantity of such small objects having both the first and second properties, and the quantity is mixed with a second quantity of other small objects that do not have both the first and second properties, and in which directional motion of the objects in the first quantity is enhanced relative to the second quantity of small objects.
